(19) United States
(12) Patent Application Publication
(10) Pub. No.: US 2021/0339291 A1
(43) Pub. Date: Nov. 4, 2021

(54) DEVICES AND METHODS FOR CLEANING EXERCISE MATS

(71) Applicant: Bryan Allan, Toronto (CA)

(72) Inventor: Bryan Allan, Toronto (CA)

(21) Appl. No.: 17/244,344

(22) Filed: Apr. 29, 2021

Related U.S. Application Data
(60) Provisional application No. 63/017,284, filed on Apr. 29, 2020.

(51) Int. Cl. B08B 3/02; B08B 1/00; B08B 11/02; B08B 1/02; B08B 7/00

(52) U.S. Cl. CPC B08B 3/022; B08B 1/002; A63B 21/4037; B08B 1/02; B08B 7/0057; B08B 11/02

(57) ABSTRACT

Devices and methods for cleaning an exercise mat are disclosed herein. The device includes a housing and a first pair of rollers positioned within the housing. The first pair of rollers are configured to receive the exercise mat through an inlet slot of the housing and direct the exercise mat towards an outlet slot of the housing. The device also includes a first sprayer positioned downstream of the first pair of rollers. The first sprayer is configured to apply a first fluid to at least one of two opposed surfaces of the exercise mat. The device also includes a brush positioned downstream of the first sprayer. The brush impacts the exercise mat to remove contaminants from the exercise mat after the first sprayer has applied the first fluid. The device also includes an ultraviolet light source configured to direct ultraviolet light onto the two opposed surfaces to disinfect each.

### CROSS-REFERENCE

This application claims priority to U.S. Provisional Application No. 63/017,284 entitled Devices and Methods for Cleaning Exercise Mats filed Apr. 29, 2020, the contents of which are incorporated herein by reference in their entirety.

### TECHNICAL FIELD

The embodiments disclosed herein relate to methods and devices for cleaning mats, and, in particular to methods and devices for cleaning exercise mats.

### BACKGROUND

As part of several exercise routines, including but not limited to yoga, participants place a thin, flexible mat on the floor directly below where they will be exercising. The mat is typically about six feet long or longer, two feet wide or wider and is typically made from a polymer-based foam or vinyl. More earth-friendly options include natural and recycled rubber, jute, and organic cotton or natural cotton (i.e. the fabric is not treated with synthetic finishes during manufacturing). These materials can create friction to facilitate the user in performing various moves and exercises. Sometimes, these mats are provided to users by a gym, school, yoga wellness studio, condominium, hotel and/or more. In other cases, some participants own their own mat.

The exercise routines noted above can be strenuous and cause the body to sweat. This sweat can be transferred to the mat during the user's workout. Sweat contains germs, bacteria, and human bodily wastes and the user's sweat can accumulate on the mat.

Further to the above, mats are generally placed on the floor and there is the potential for cross-contamination between the floor (e.g. public spaces, gym facilities, etc.) and the user's hands and/or feet and/or faces. Therefore, there is the potential for bacteria and viruses to travel between the floor to the user's hands, feet and face very quickly. As a result, user's typically desire mats to be cleaned and sanitized either before or after a workout.

Accordingly, there is a need for methods and devices for cleaning exercise mats.

### SUMMARY

In one broad aspect, a device for cleaning an exercise mat is described herein. The device includes a housing; a first pair of rollers positioned within the housing and being configured to receive the exercise mat through an inlet slot of the housing and direct the exercise mat towards an outlet slot of the housing; a first sprayer configured to apply a first fluid to at least one side of the exercise mat as the exercise mat passes between the inlet slot of the housing and the outlet slot of the housing; a brush configured to manually impact the exercise mat to remove contaminants from the exercise mat as the exercise mat passes between the inlet slot of the housing and the outlet slot of the housing; an ultraviolet source configured to direct ultraviolet light onto at least a portion of the exercise mat to disinfect the at least a portion of the exercise mat as the exercise mat passes between the inlet slot of the housing and the outlet slot of the housing; a second sprayer configured to spray a second fluid onto at least a portion of the exercise mat as the exercise mat passes between the inlet slot of the housing and the outlet slot of the housing; and a processor. The processor includes a communication interface configured to communicate with at least one computing device via a communication network; a storage unit configured to store data for the device; and a processing unit configured to receive instructions from the communication interface and control the first sprayer, the brush, the ultraviolet source and/or the second sprayer based on the instructions to clean the exercise mat.

In at least one embodiment, the device also includes a second set of rollers positioned within the housing downstream from the first set of rollers, the second set of rollers being configured to receive the exercise mat from the first set of rollers and direct the exercise mat towards an outlet slot of the housing.

In at least one embodiment, the processing unit is further configured to receive a payment authorization from the communication interface and control the first sprayer, the brush, the ultraviolet source and/or the second sprayer based on the payment authorization to clean the exercise mat.

In at least one embodiment, the first sprayer is positioned downstream of the first pair of rollers within the housing.

In at least one embodiment, the first sprayer includes one or more nozzles positioned to direct a fluid onto the exercise mat at differing angles.

In at least one embodiment, the first sprayer is positioned to direct a fluid onto the exercise mat as the exercise mat is bent to expose grooves of the exercise mat and provide for the first fluid to enter the grooves of the exercise mat.

In at least one embodiment, the first fluid is a disinfectant.

In at least one embodiment, the brush is positioned downstream of the first sprayer within the housing.

In at least one embodiment, the brush is positioned upstream of the first sprayer within the housing.

In at least one embodiment, the UV source is positioned downstream of the brush within the housing.

In at least one embodiment, the UV source includes one or more UV lamps configured to disinfect at least one surface of the exercise mat.

In at least one embodiment, the UV source includes one or more UV lamps configured to disinfect two surfaces of the exercise mat.

In at least one embodiment, the second sprayer is positioned downstream of the ultraviolet source within the housing.

In at least one embodiment, the second sprayer is configured to apply the second fluid to two surfaces of the exercise mat.

In at least one embodiment, the second fluid is a scent.

In another broad aspect, a method for cleaning an exercise mat is described herein. The method includes selecting, via a mobile application of a mobile device, a cleaning methodology of a cleaning device from a plurality of cleaning methodologies of the cleaning device; sending, via a wireless network, the selected cleaning methodology from the mobile device to the cleaning device; and inserting an exercise mat into the cleaning device for the cleaning device to perform the selected cleaning methodology on the exercise mat.

In at least one embodiment, the selecting, via a mobile application of a mobile device, the cleaning methodology of the cleaning device includes selecting one or more cleaning functions of the cleaning device.

In at least one embodiment, the one or more cleaning functions include selecting a disinfecting solution of the cleaning device to be applied to the exercise mat.

In at least one embodiment, the one or more cleaning functions include selecting a scent of the cleaning device to be applied to the exercise mat.

In at least one embodiment, the one or more cleaning functions include selecting a number of sides of the exercise mat to be cleaned.

In at least one embodiment, the sending, via a wireless network, the selected cleaning methodology from the mobile device to the cleaning device includes sending a payment authorization from the mobile device to the cleaning device.

In another broad aspect, a device for cleaning an exercise mat, the device comprising: a housing having: a main body having an inlet slot configured to receive the exercise mat and an outlet slot; and a door having a concave shape and being hingedly coupled to the main body to be movable between a closed position where the door covers the outlet slot and an open position where the outlet slot is uncovered.

In at least one embodiment, the main body includes an upper portion configured to support the exercise mat when the exercise mat is in a rolled configuration.

In at least one embodiment, the upper portion has a concave shape and extends across a width of the main body to support the exercise mat when the exercise mat is in a rolled configuration.

In at least one embodiment, the inlet slot is positioned nearest to a rear side of the housing to receive an end of the exercise mat when the exercise mat is supported by the upper portion of the main body in the rolled configuration.

In at least one embodiment, the door is positioned on a bottom side of the housing.

In at least one embodiment, the door is hingedly coupled to the housing nearest to a rear side of the main body.

In at least one embodiment, the door is configured to receive and support the exercise mat as the exercise mat exits the main body.

In at least one embodiment, the door has a concave shape and extends across a width of the main body to receive and support the exercise mat as the exercise mat exits the main body and to guide the exercise mat to return to the rolled configuration thereon.

According to a broad aspect, a device for cleaning an exercise mat is described herein. The device comprises a housing and a first pair of rollers positioned within the housing and configured to receive the exercise mat through an inlet slot of the housing and direct the exercise mat towards an outlet slot of the housing; a first sprayer positioned downstream of the first pair of rollers and configured to apply a first fluid to at least one of two opposed sides of the exercise mat as the exercise mat passes between the inlet slot of the housing and the outlet slot of the housing. The device also includes a brush positioned downstream of the first sprayer and configured to impact the exercise mat to remove contaminants from the exercise mat after the first sprayer has applied the first fluid as the exercise mat passes between the inlet slot of the housing and the outlet slot of the housing. The device also includes an ultraviolet light source configured to direct ultraviolet light onto a first surface and a second surface of the two opposed surfaces of the exercise mat to disinfect each of the first surface and the second surface after the brush has impacted the exercise mat.

In at least one embodiment, the device also includes a storage reservoir to store the first fluid and a pump to provide the first fluid from the storage reservoir to the first sprayer.

In at least one embodiment, the pump is communicatively connected to a door covering the inlet slot such that the pump is activated to provide the first fluid to the first sprayer when the door to the inlet slot is open.

In at least one embodiment, the pump is communicatively connected to the door covering the inlet slot such that the pump is deactivated when the door to the inlet slot is closed.

In at least one embodiment, the device also includes a second set of rollers positioned within the housing downstream from the first set of rollers, the second set of rollers being configured to receive the exercise mat from the first set of rollers and direct the exercise mat towards an outlet slot of the housing.

In at least one embodiment, the device also includes a processor comprising a communication interface and a processing unit configured to receive instructions from the communication interface and control the first sprayer, the brush and/or the ultraviolet source based on the instructions from the communication interface.

In at least one embodiment, the processing unit is configured to, in response to receiving a payment authorization from the communication interface, activate the first sprayer, the brush and/or the ultraviolet source.

In at least one embodiment, the communication interface is configured to transmit a payment request to a payment server and receive the payment authorization from the payment server.

In at least one embodiment, the first sprayer includes one or more nozzles positioned to direct the first fluid onto the exercise mat at differing angles.

In at least one embodiment, the first sprayer is positioned to direct a fluid onto the exercise mat as the exercise mat is bent to expose grooves of the exercise mat and provide for the first fluid to enter the grooves of the exercise mat.

In at least one embodiment, the first fluid is a disinfectant.

In at least one embodiment the device also includes a tray positioned below the first sprayer, the tray configured to collect at least a portion of the first fluid.

In at least one embodiment, the tray is removable from the housing.

In at least one embodiment, the storage reservoir is positioned outside of the housing.

In at least one embodiment, the storage reservoir is positioned inside of the housing.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is front perspective view from above of a device for cleaning exercise mats, according to at least one embodiment.

FIG. 2 is a front perspective view from above of the device for cleaning exercise mats shown in FIG. 1 with an exit door on a front of the device in an open position.

FIG. 3 is a front perspective view from above of another device for cleaning exercise mats with an exit door on a bottom of the device in an open position, according to at least one embodiment.

FIG. 4 is a rear perspective view of the device for cleaning exercise mats shown in FIG. 3.

FIG. 5 is a front view of the device for cleaning exercise mats shown in FIG. 3 with the exit door on the bottom of the device in a closed position.

FIG. 6A shows a front perspective view from above of the device for cleaning exercise mats of FIG. 3 with the exit door in the closed position.

FIG. 6B shows a front perspective view from above of the device for cleaning exercise mats of FIG. 3 with an exercise mat loaded onto the device.

FIG. 6C shows a front perspective view from above of the device for cleaning exercise mats of FIG. 3 with an exercise mat loaded onto the device and the exit door in the open position.

FIG. 6D shows a front perspective view from above of the device for cleaning exercise mats of FIG. 3 with the exit door in the open position and the exercise mat passing through the device.

FIG. 6E shows a front perspective view from above of the device for cleaning exercise mats of FIG. 3 with the exit door in the open position and the cleaned exercise mat being supported by the exit door.

FIG. 6F shows a front perspective view from above of the device for cleaning exercise mats of FIG. 3 with the exit door in the open position.

FIG. 7 is a right side view of a device for cleaning exercise mats with a side panel of the device removed and an exit door in a closed position, according to at least one embodiment.

FIG. 8 is a right side view of a device for cleaning exercise mats with a side panel of the device removed and an exit door in the open position showing a pathway of an exercise mat through the device, according to at least one embodiment.

FIG. 9 is a schematic left side view of a device for cleaning exercise mats, according to at least one embodiment.

FIG. 10 is a partial front perspective view from a left side of two sets of rollers of a device for cleaning exercise mats, according to at least one embodiment.

FIG. 11 is a partial front perspective view from a right side of two sets of rollers of a device for cleaning exercise mats, according to at least one embodiment.

FIG. 12 is a partial side perspective view of a device for cleaning exercise mats without a housing, according to at least one embodiment.

FIG. 13 is a picture showing a side view of a device for cleaning exercise mats with its housing removed, according to at least one embodiment.

FIG. 14 is a front perspective view of a sprayer of a device for cleaning exercise mats, according to one embodiment.

FIG. 15 is a front perspective view of a portion of an internal mechanism of a device for cleaning exercise mats, according to one embodiment.

FIG. 16 is a front perspective view of a device for cleaning exercise mats with its housing partially removed showing a processor thereof, according to at least one embodiment.

FIG. 17 is a block diagram of a processor and other components of a device for cleaning exercise mats, according to at least one embodiment.

FIG. 18 is a method of cleaning exercise mats, according to at least one embodiment.

FIG. 19 is a is a front perspective view of a device for cleaning exercise mats with its housing partially removed showing a processor thereof, according to at least one embodiment.

FIG. 20 is another front perspective view of a device for cleaning exercise mats with its housing partially removed showing a processor thereof, according to at least one embodiment.

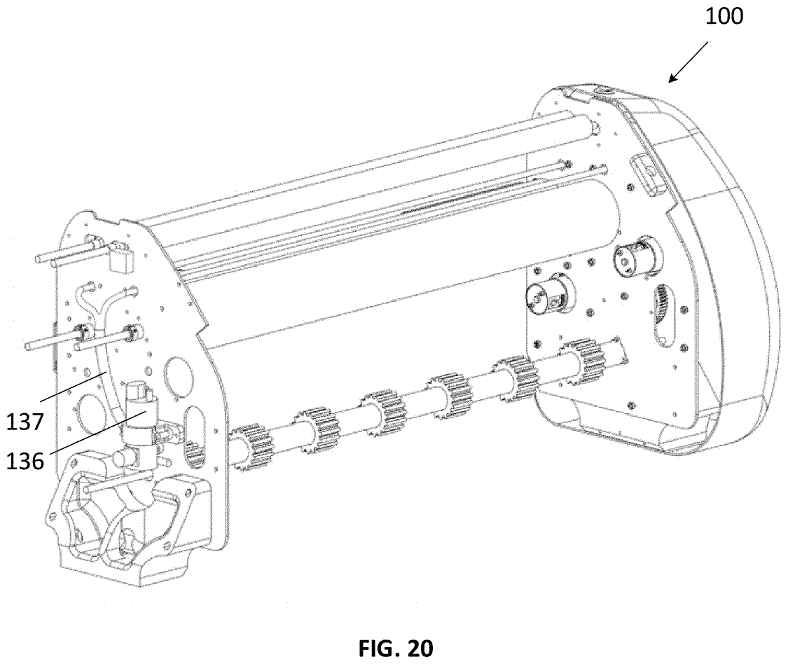

FIG. 21 is a side view of the device of FIG. 19.

FIG. 22 is another side view of the device of FIG. 19.

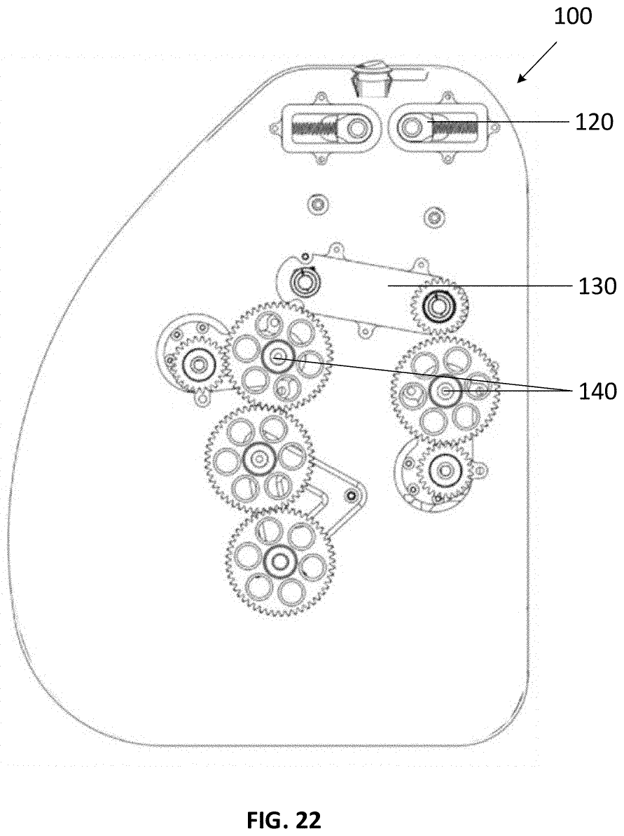

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

Terms of degree such as “about” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Some of the embodiments of the devices and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers may be a server, network appliance, embedded device, computer expansion module, a personal computer, laptop, personal data assistant, cellular telephone, smart-phone device, tablet computer, a wireless device or any other computing device capable of being configured to carry out the methods described herein.

In at least one embodiment, the devices described herein may include a processor having a communication interface and a processing unit. The communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof. In at least one embodiment, the communication interface may provides the processor with connectivity to one or more communication links and/or networks (e.g., LANs, WANs, or wireless LANs) by which the processor may communicate with other devices (e.g. user devices and/or remote servers and/or computing devices).

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein.

Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer usable instructions may also be in various forms, including compiled and non-compiled code.

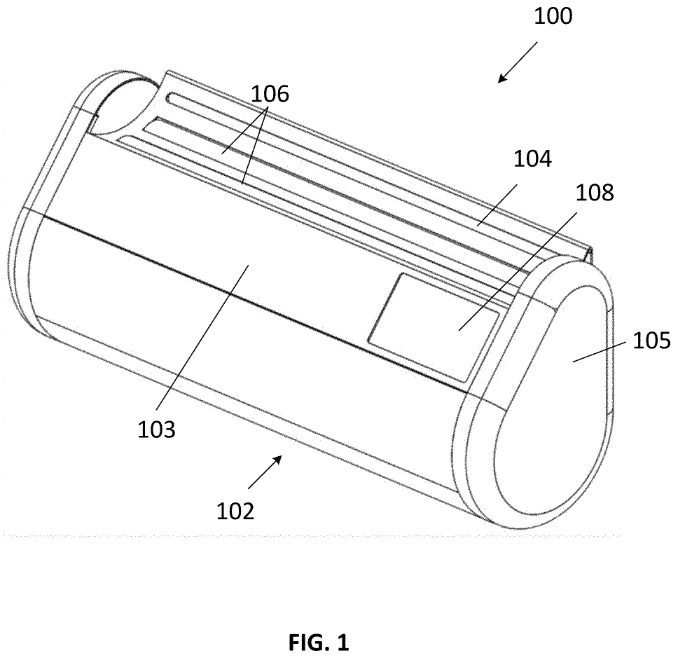

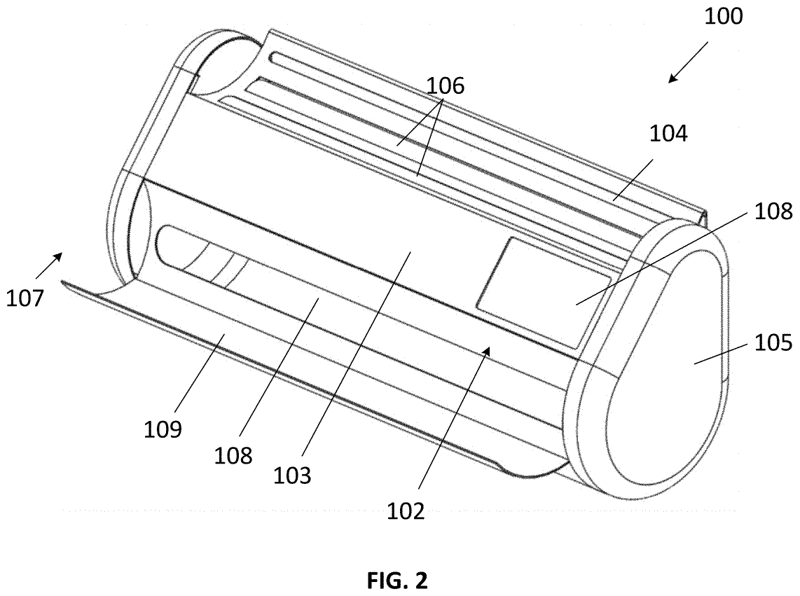

Turning to the drawings, FIG. 1 shows a cleaning device 100 having a housing 102 including a main body 103, two side panels 105 on either side of the body 103, and, optionally, a door 109 (see FIG. 2) hingedly connected to at least one of the body 103 and the side panels 105. Body 103 includes an inlet slot 104 and an outlet slot 108 (see FIG. 2). In at least one embodiment, the inlet slot 104 is positioned rearwardly of guide rollers (not shown) positioned in slots 106. The guide rollers reduce friction between the exercise mat (not shown) being fed into the inlet slot 104. FIG. 2 shows door 109 of the device 100 in an open position.

In at least one embodiment, main body 103 also includes a display 108. One embodiment of display 108 is shown on FIGS. 1, 2 and 4. Display 108 may be an LCD display or the like. Display 108 may present a user interface to a user of the device 100. Display 108 may also be used as an input device for a user of the device 100 to input selections presented to the user. For instance, the user interface may present different cleaning features and/options that are available on the device 100. For instance, the user interface may present different cleaning functions to be applied to the user's exercise mat via display 108, such as but not limited to: application of a disinfectant by a first sprayer 130 (as described below), activation of one or more brushes, activation of one or more ultraviolet (UV) sources and application of a scent. In another example, the user interface may present different cleaning options to be applied to the user's exercise mat via display 108, such as but not limited to: a choice of one type of disinfectant to be applied to the exercise mat and/or a choice of one type of scent to be applied to the exercise mat. In at least one embodiment, display 108 may be a touchless display. For instance, display 108 may be used as a touchless input device for a user to initiate a payment transaction (e.g. a near field communication (NFC) enabled point-of-sale terminal) with a NFC enabled device such as but not limited to a NFC enabled mobile device, a NFC enabled payment card or the like.

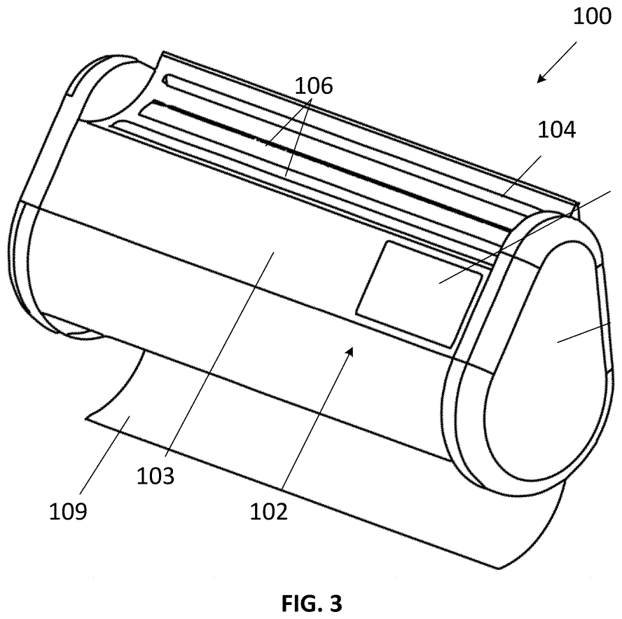

Door 109 may be positioned on a front side 107 of device 100 as shown in FIG. 2. Door 109 is generally movable between a closed position (see FIG. 1) and an open position (see. FIG. 2). When in the open position, outlet slot 108 is viewable in the main body 103. Outlet slot 108 is sized and shaped to provide for an exercise mat to pass therethrough once it has been cleaned in the cleaning device 100. Outlet slot 108 may be positioned on front side 107 of device 100 or, as shown in FIG. 3, on bottom side 111 of device 100.

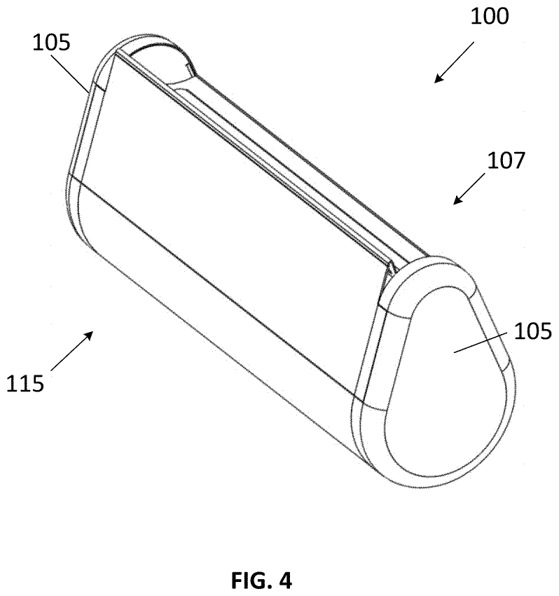

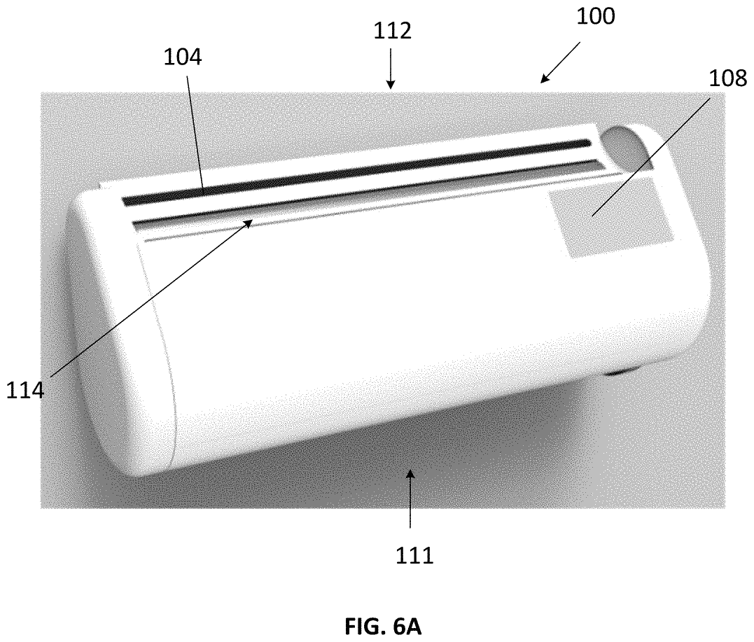

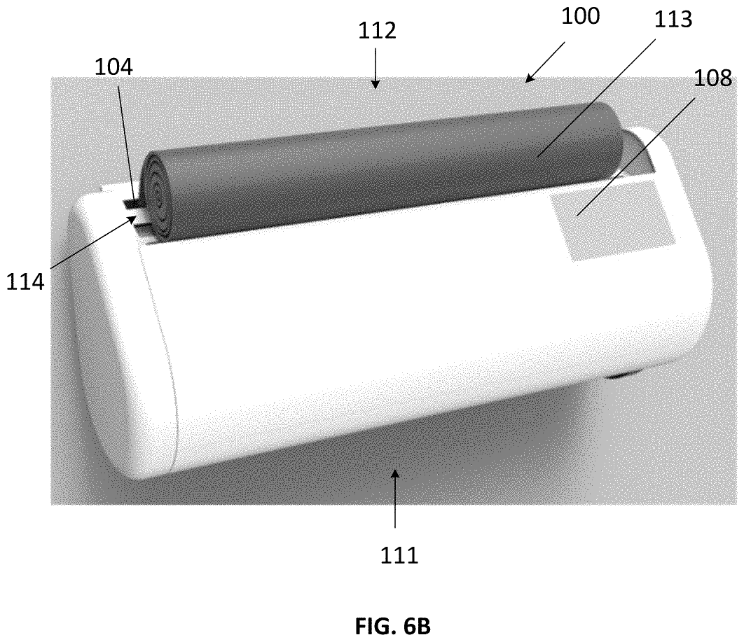

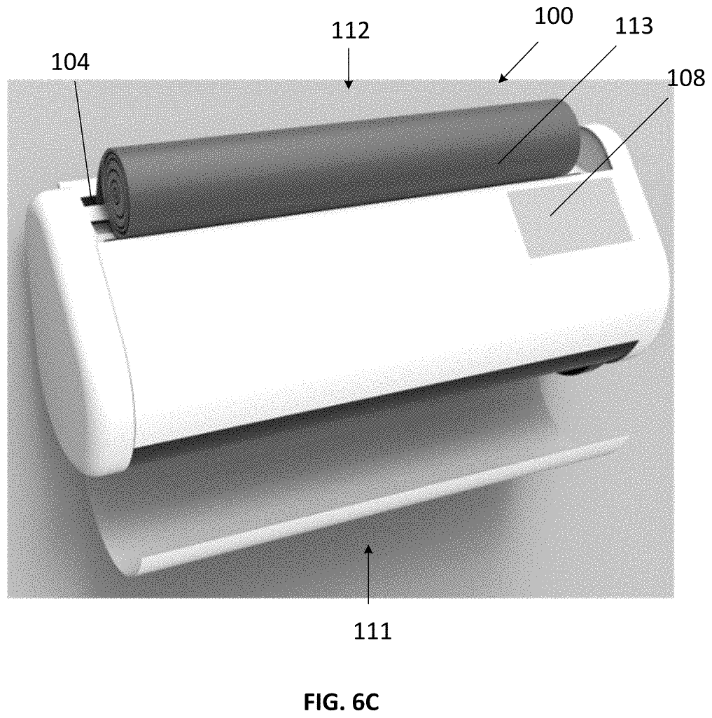

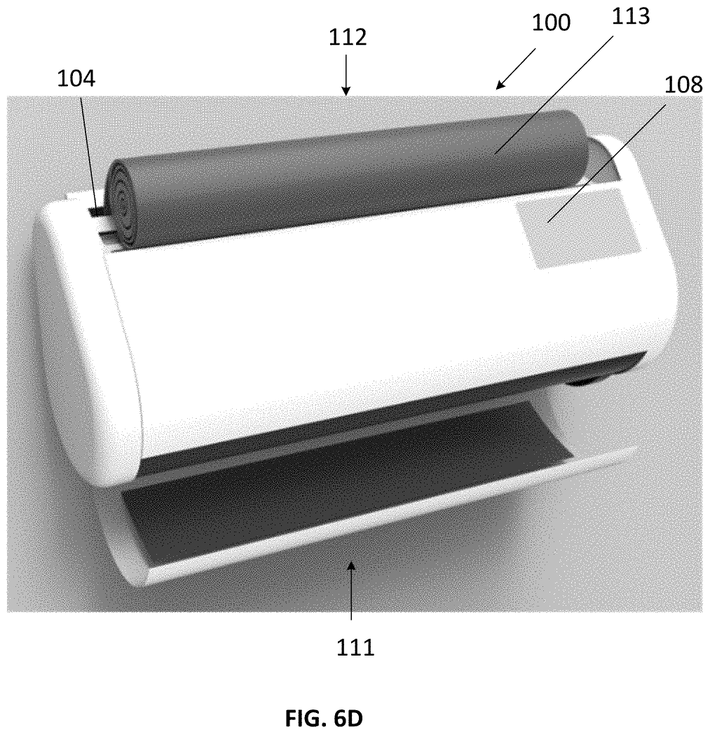

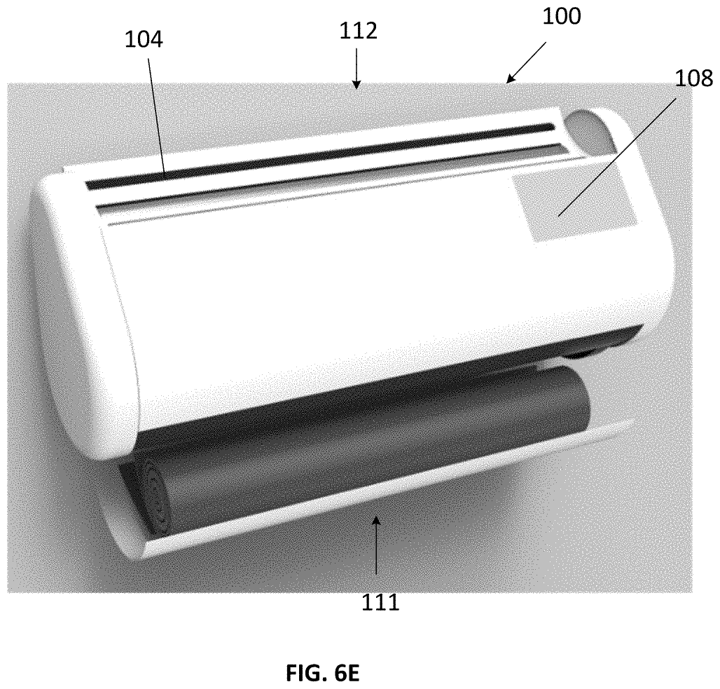

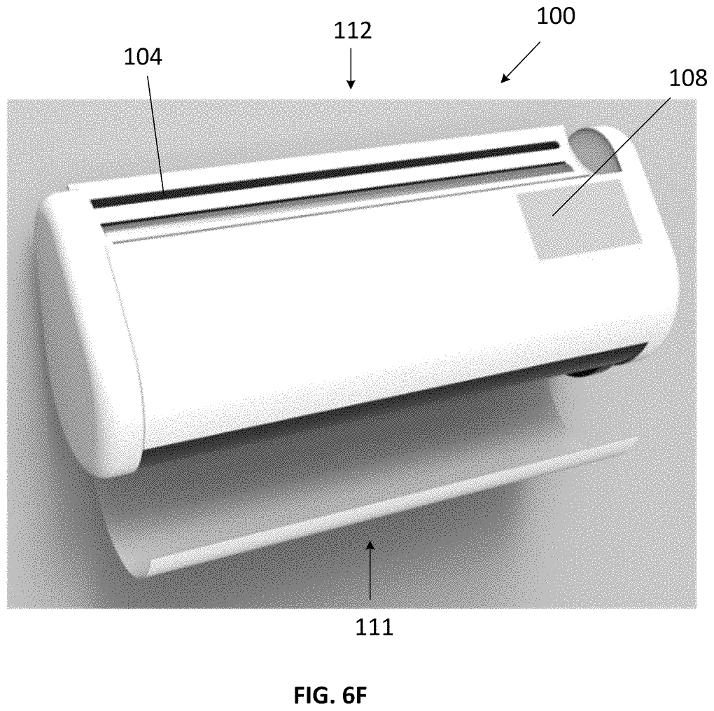

FIG. 3 shows another embodiment of the device 100 showing door 109 being positioned on a bottom side 111 of the device 100. Bottom side 111 of the device 100 is opposed to the top side 112 having inlet slot 104. In this embodiment, it should be noted that the door 109 may be configured to receive and support the exercise mat as the exercise mat exits the device 100. Further, it should be noted that inlet slot 104 may be positioned nearer to a rear side 115 (see FIG. 4) than to front side 107. Positioning the inlet slot 104 nearer to the rear side 115 than to the front side 107 may provide for inlet slot to 104 to receive an exercise mat when it is rolled up and being supported on an upper portion 114 of the body 103 (see FIG. 6B).

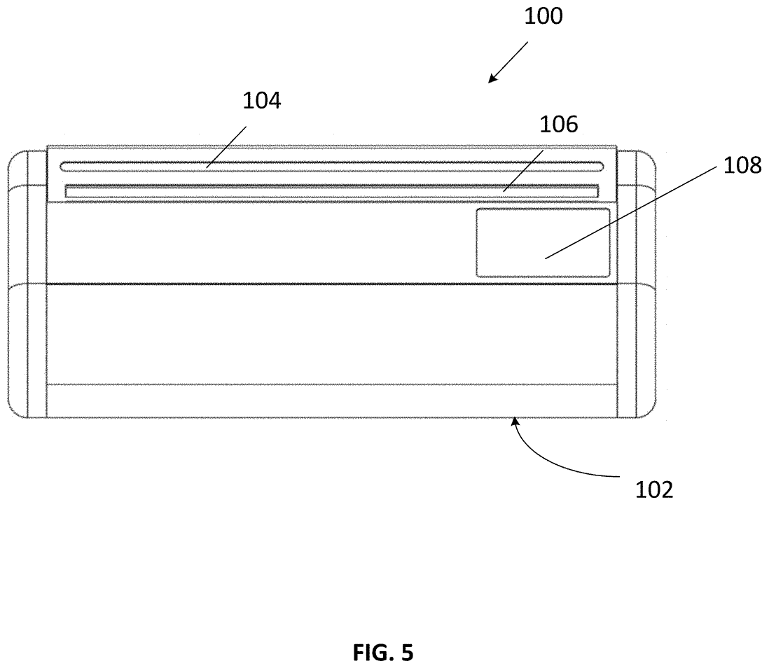

FIG. 5 shows a front view of the device 100 for cleaning exercise mats shown in FIG. 3 with the exit door 109 on the bottom side 111 of the device 100 in a closed position.

FIGS. 6A-6F show steps of a process of cleaning an exercise mat by passing the exercise through the device 100 shown in FIG. 3. Specifically, FIG. 6A shows a front perspective view from above of device 100 with the exit door 109 in the closed position. Here, the device is ready to receive instructions from a user (e.g. via display 108).

FIG. 6B shows a front perspective view from above of the device 100 after a user has placed an exercise mat 113 on an upper portion 114 of the body 103. Upper portion 114 of the body 103 extends across the top side 112 of the body 102 and has a concave shape to receive exercise mat 113 and support exercise mat 113 thereon when the exercise mat 113 is rolled (i.e. in a rolled configuration).

FIG. 6C shows device 100 supporting an exercise mat 113 and having the door 109 in its open position. At this stage, the device 100 is ready to begin cleaning the exercise mat 113. It should be noted that the device 100 is configured to automatically draw the exercise mat 113 inwardly (e.g. into housing 103) when it has received instructions and/or payment from a user provided that the user has inserted at least a portion of the exercise mat 113 into inlet slot 104.

FIG. 6D shows device 100 and the exercise mat 113 having partially passed therethrough.

FIG. 6E shows device 100 after the exercise mat 113 has been cleaned and passed through the device 100. Here, exercise mat is shown being supported on the door 109. In at least one embodiment, door 109 has a concave shape to receive the exercise mat 113 as it passes through the outlet slot 108. In at least one embodiment, the concave shape of door 109 provides for the exercise mat 113 to roll up (i.e. return to a rolled configuration) as it passes through the outlet slot 108 and exits housing 102.

FIG. 6F shows a device 100 with door 109 in the open position after a user has removed the exercise mat 113 therefrom.

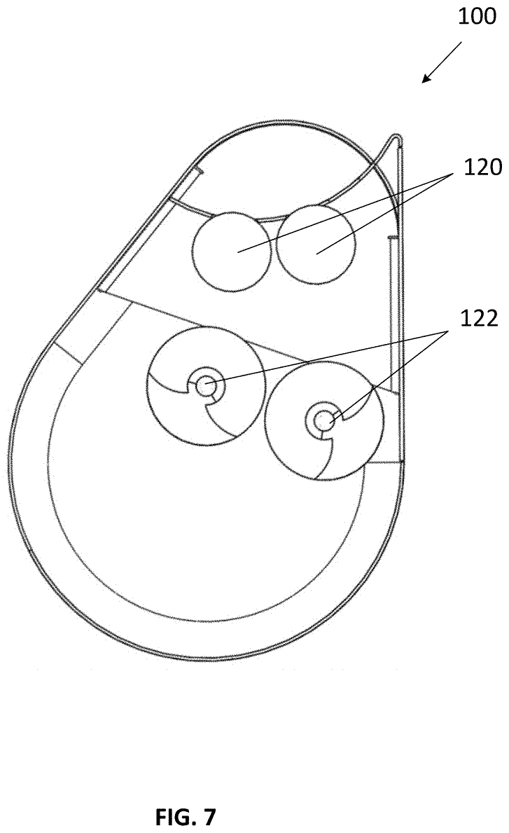

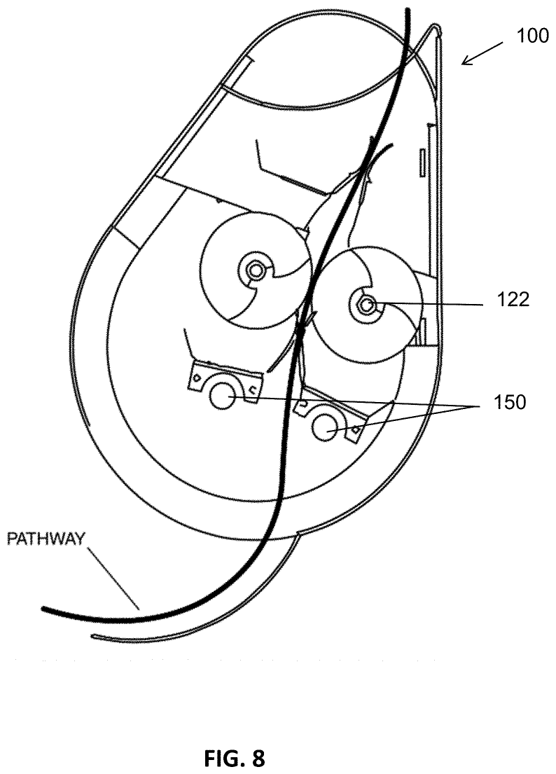

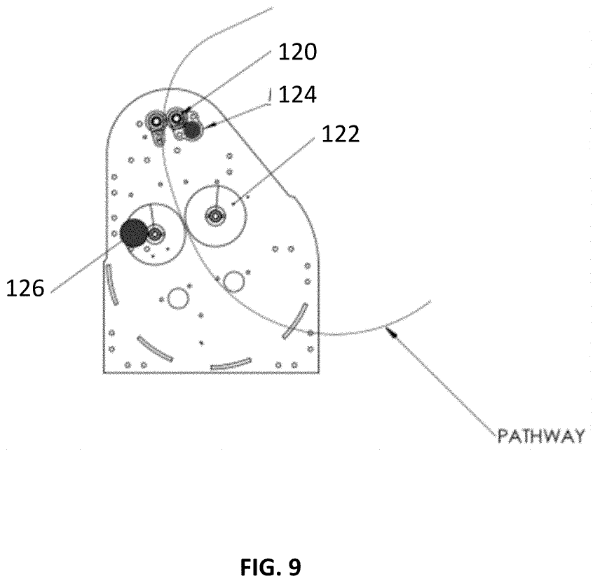

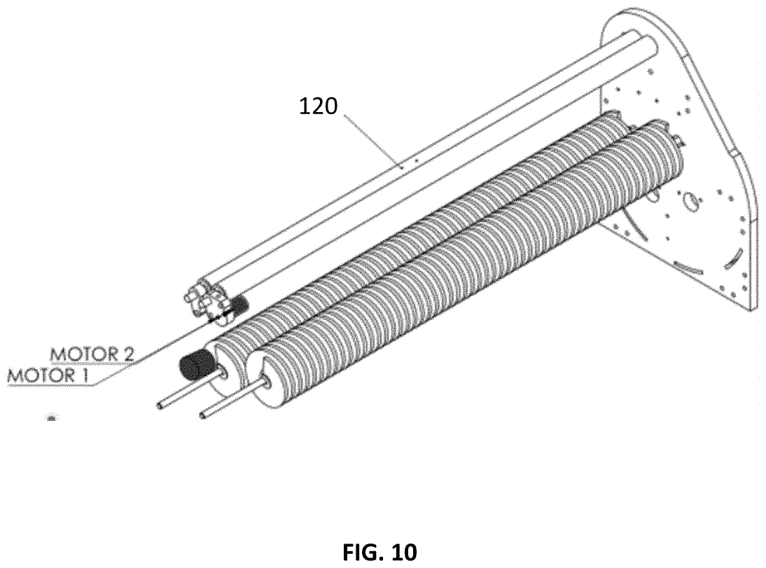

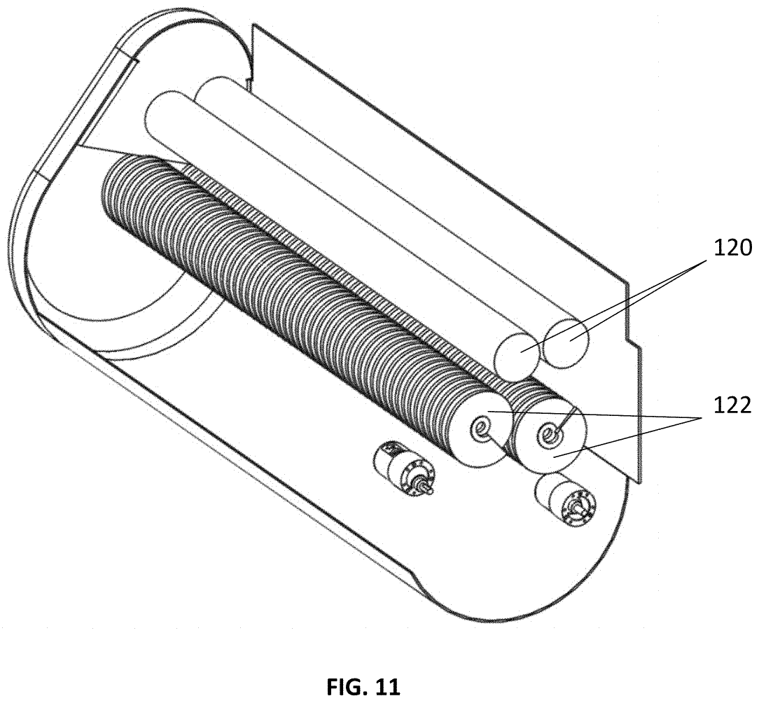

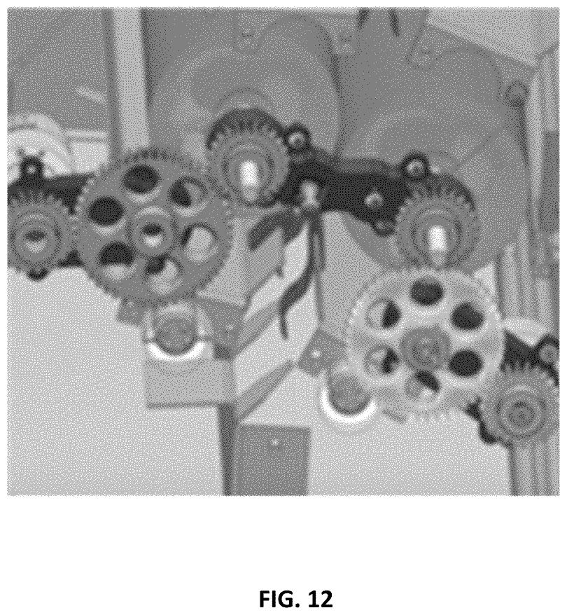

As shown in FIGS. 7 to 12, in at least one embodiment, housing 102 includes a first pair of rollers 120 to receive an exercise mat (e.g. exercise mat 113) through the inlet slot 104 of the housing 102 and direct the exercise mat towards the outlet slot 108 of the housing 102.

In at least one embodiment, housing 102 may include a second pair of rollers 122 to receive the exercise mat from the first set of rollers 120 and direct the exercise mat towards an outlet slot 108 of the housing 102. In at least one embodiment, housing 102 may include more than two sets of rollers. Each set of rollers may be driven by a motor, such as motors 124 and 126, respectively, as shown in FIGS. 7, 8, 9, 10, 11 and/or 12. In at least one embodiment, the first pair of rollers 120 include a sensor (not shown) that is activated by the exercise mat when it is inserted by the user into the housing 102. Activation of the sensor may activate the at least one or the motors 124, 126 to receive the exercise mat and automatically pull the exercise mat inwardly. In this manner, the cleaning device 100 may be referred to as being “auto-feeding” or “self-feeding”.

Housing 102 may also include one or more guides (not shown) for directing the exercise mat as it passes through the device 100. One or more guides may be positioned anywhere within the housing 102 along a pathway of the exercise mat between the inlet 104 and the outlet 108 to direct the exercise mat along the pathway. For instance, the one or more guides may be positioned between the inlet slot 104 and the first set of rollers 120, between the first set of rollers 120 and a second set of rollers 122, and/or between the second set of rollers 122 and the outlet slot 108. In embodiments where there are more than two set of rollers, the guides may be positioned anywhere within the housing 102 along a pathway of the exercise mat between the inlet 104 and the outlet 106.

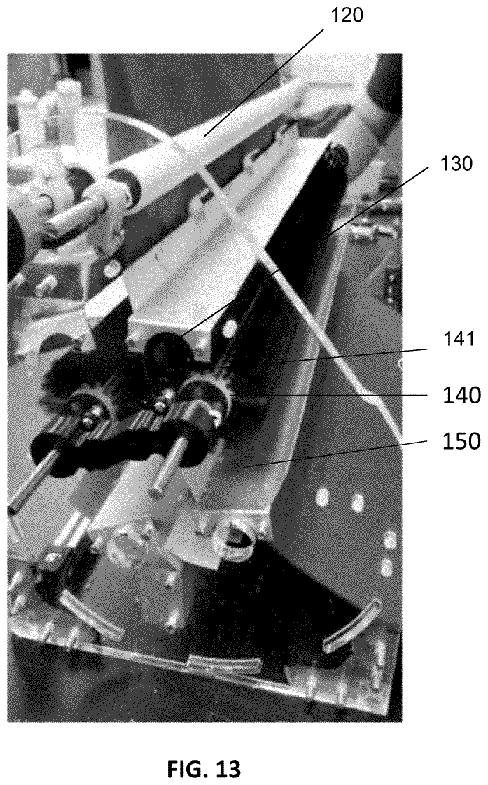

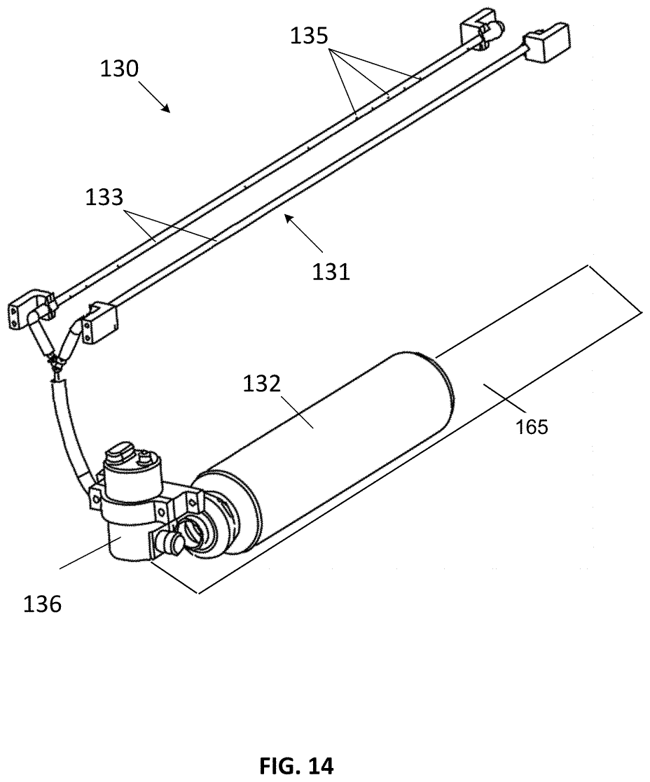

Housing 102 also includes a first sprayer 130 (see FIGS. 13 and 14) positioned downstream of the first pair of rollers 120. The first sprayer 130 is coupled to the housing 102 (e.g. directly or indirectly) and configured to apply one or more fluids, such as but not limited to water and/or a disinfectant or a disinfectant solution, to at least a portion of at least one side of the exercise mat as the exercise mat longitudinally passes along the pathway between the inlet slot 104 of the housing 102 and the outlet slot 108 of the housing 102.

In at least one embodiment, the first sprayer 130 as shown in FIG. 14 is configured to apply (e.g. spray) a stream of fluid (e.g. liquid or gas) onto at least a portion of the exercise mat to be cleaned. For instance, at least a portion of the first sprayer 130 may be configured to have a width that is equal to or greater than a typical width of an exercise mat (e.g. about two feet, or 24 inches) and spray a linear stream of fluid across a width of the exercise mat as the exercise mat longitudinally passes the first sprayer 130.

In another embodiment, the first sprayer 130 may be configured to have a width that is less than a typical width of an exercise mat and spray a linear stream of fluid across a portion of the width of the exercise mat as the exercise mat longitudinally passes the first sprayer 130. In this embodiment, the first sprayer 130 may be movably coupled to the housing 100 and be configured to move laterally in a direction across the width of the exercise mat as the exercise mat passes longitudinally along the pathway.

In the embodiment shown in FIG. 14, the sprayer 130 includes a nozzle portion 131 including two nozzle heads 133, each including a plurality of nozzles 135 (i.e. apertures) that are spread at least across a width of an exercise mat to spray a stream of fluid onto two surfaces of the exercise mat (e.g. front and back surfaces) as the exercise mat longitudinally passes the two nozzle heads 133. Each of the two nozzle heads 133 is fluidly coupled to reservoir 132 and configured to receive fluid from the reservoir 132. In at least one embodiment, nozzles 135 are evenly spaced from each other along each of the nozzle heads 133.

In at least one embodiment, each of the one or more nozzle heads 133 is configured to apply one or more fluids to the exercise mat simultaneously. For instance, the one or more nozzle heads 133 may be positioned to apply the one or more fluids to the exercise mat at different angles and from different distances. In at least one embodiment, varying the number of nozzles 135 along one or more of the nozzle heads 133 and/or varying the distance of nozzles 135 along one or more of the nozzle heads 133 from the exercise mat may vary the contact angle of the fluid as it strikes the exercise mat, which may optimize the coverage of the fluid on the exercise mat.

For instance, in at least one embodiment, one or more nozzle heads 133 of the first sprayer 130 may be positioned along the pathway at a position where directing the fluid onto the exercise mat will provide for the fluid to strike a surface of the exercise mat at a position when the exercise mat is bent along the pathway. In this manner, the fluid applied to the exercise mat may be able to penetrate small grooves of the exercise mat that are widened when the exercise mat is bent along the pathway.

As noted above, the fluid to be applied to the exercise mat as the exercise mat travels along the pathway may be, but is not limited to, water, a disinfectant and/or a cleaning solution. In at least one embodiment, the fluid may be a cleaning solution such as but not limited to a green cleaning product(s) that cleans the exercise mat naturally, without any unpleasant chemical residue that may harm the user of the exercise mat. In at least one embodiment, green cleaning products as contemplated herein may be eco-friendly cleaning products typically made using sustainable manufacturing practices and naturally-derived, safe, non-toxic, and biodegradable ingredients that don't negatively impact the environment or the user's health.

In at least one embodiment, first sprayer 130 may be fluidly coupled to reservoir 132 configured to retain the one or more fluids to be applied to the exercise mat. The one or more fluids may be stored as a liquid and may be applied to the mat as a liquid, or, in at least one embodiment, may be aerosolized by first sprayer 130 and applied to the exercise mat as a gas. First sprayer 130 therefore generally also includes a pump (e.g. pump 136 of FIG. 14) that either controls the delivery of the fluid to the mat or receives instructions for the delivery of the fluid to the exercise mat from the processor 170.

In at least one embodiment, first sprayer 130 and/or pump 136 is communicatively coupled to the processor 170 (described below) to receive control signals from the processor 170. For instance, pump 136 may be configured to receive a control signal from the processor 170 activating the pump 136 to transfer the first fluid from the reservoir 132 to the first sprayer 130 in response to a stimulus. For instance, pump 136 may be configured to receive a control signal from the processor 170 activating the pump 136 to transfer the first fluid from the reservoir 132 to the first sprayer 130 in response to a sensor indicating that a mat has been inserted into inlet slot 104. In another embodiment, one or more doors covering inlet slot 104 may be communicatively coupled to the pump 136 such that opening one or more of the doors (e.g. by inserting an exercise mat into inlet slot 104) may activate a switch that is coupled to pump 136 and activates pump 136 to transfer the first fluid from the reservoir 132 to the first sprayer 130. Similarly, as the mat passes through the first pair of rollers 120, the doors covering the inlet slot may close and deactivate the pump 136.

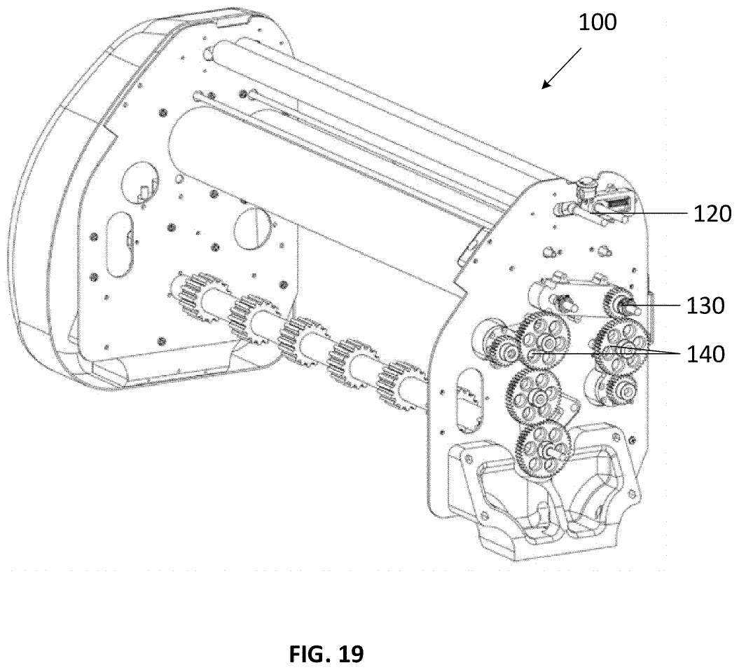

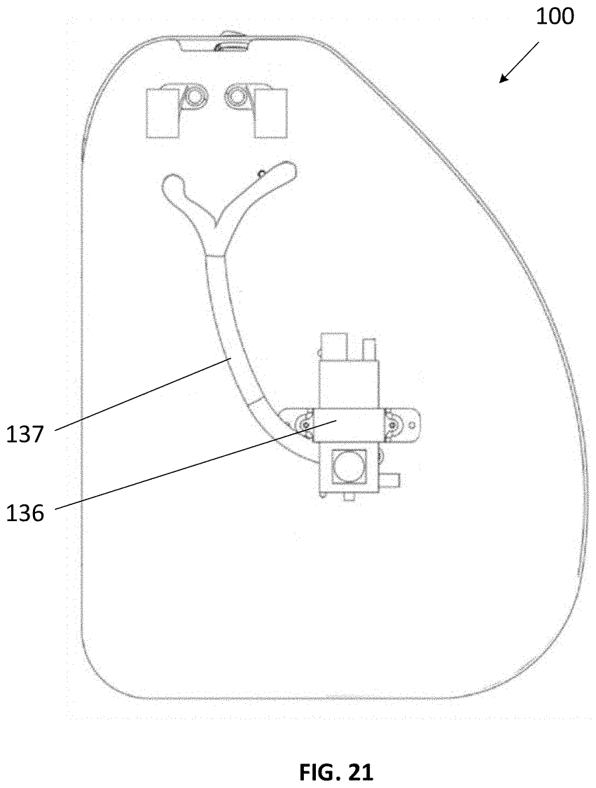

In at least one embodiment, reservoir 132 may be positioned within the housing 100 and be fluidly coupled to the first sprayer 130 by tubing (see for example FIG. 21). In at least one embodiment, reservoir 132 may be positioned outside the housing 102 and be fluidly coupled to the first sprayer 130 by tubing (see for example FIG. 19). Positioning the reservoir 132 outside of housing 102 may provide for the reservoir 132 to be refilled without having to access an internal compartment of the housing 102 (e.g. by removing or opening a portion of the housing 102).

In at least one embodiment the device 100 also includes a tray 165 (see FIG. 14), for example positioned below the first sprayer 130. Tray 165 may be configured to collect at least a portion of the first fluid as the first fluid is sprayed onto the exercise mat and, potentially, falls downwardly off of the exercise mat or, for example, bounces off of the exercise mat. Tray 165 collects excess first fluid and inhibits the first fluid from falling out of the housing 102 or collecting in a bottom portion of the housing 102. In at least one embodiment, the tray 165 is removable from the housing 102 to remove any excess fluid thereon. Tray 165 may be, in at least one embodiment, positioned towards a front of the housing 102 and be accessible by removing a front panel of the housing 102. In at least one embodiment, tray 165 may retain or support the reservoir 132.

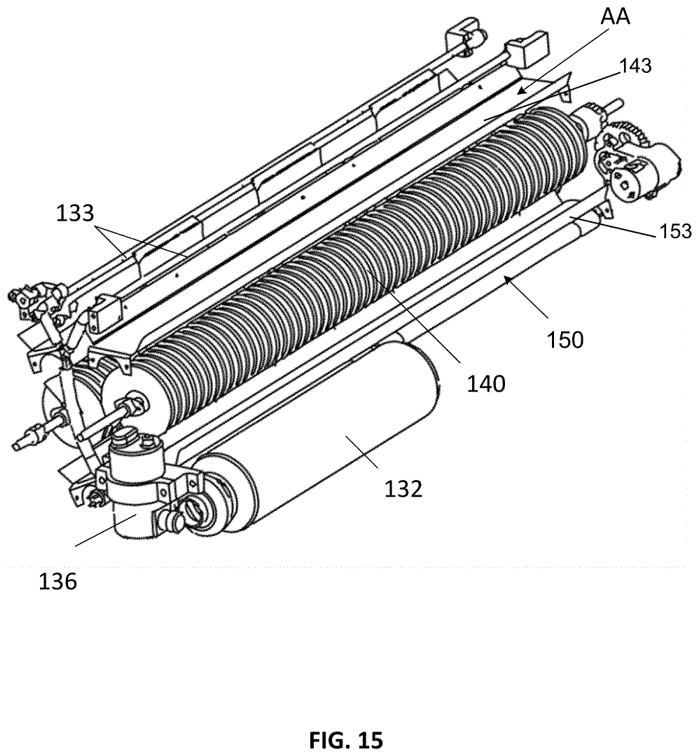

Device 100 also includes at least one brush 140 (see FIGS. 13 and 15) for mechanically agitating the exercise mat to, for example, remove debris from the exercise mat. In at least one embodiment, the at least one brush 140 is positioned downstream of the first sprayer 130 along the pathway. In at least one embodiment, the at least one brush 140 is positioned upstream of the first sprayer 130 along the pathway. In at least one embodiment, the at least one brush 140 may be positioned on an outer surface of the second set of rollers 122. In at least one embodiment, the device 100 includes two brushes 140 positioned on either side of the mat as the mat travels between the sprayer 130 and the UV light source 150, the two brushes each being configured to contact one of the two surfaces of the mat.

Brush 140 is configured to impact the exercise mat to remove or dislodge debris and/or contaminants from the exercise mat as the exercise mat passes between the inlet slot 104 of the housing 102 and the outlet slot 108 of the housing 102.

In at least one embodiment, each of the one or more brushes 140 is used to enhance the interaction of the disinfectant with the debris in the exercise mat.

For example, in at least one embodiment, brush 140 includes one or more rotating scrub brushes that have an axis of rotation substantially perpendicular to the exercise mat's surface as the exercise mat travels along the pathway. In at least one embodiment, brush 140 may include more than one rotating scrub brush 141 that are positioned on opposite sides of the exercise mat to stabilize the exercise mat. In at least one embodiment, the rotating brushes rotate in different directions to neutralize a lateral force applied by the rotating brushes to the exercise mat. Each of the scrub brushes (e.g. having bristles thereon), in at least one embodiment, extends a length about equal to or greater than a width of a typical exercise mat. (e.g. about 24 inches wide). In at least one embodiment, the brushes 140 are spaced apart from each other by a distance to provide for a typical exercise mat (e.g. having a thickness of about 4-5 mm) to pass therebetween and for the bristles of the brush heads to contact each side of the mat.

In at least one embodiment, the brush 140 includes one or more rotary brushes that have an axis of rotation essentially parallel to the exercise mat's surface as the exercise mat travels along the pathway. In these embodiments, the rotation of the rotary brush(es) can aid in moving the mat through the device 100.

Brush 140 is communicatively coupled to the processor 170 (described below) to receive control signals from the processor 170. For instance, brush 140 may be configured to receive a control signal from the processor 170 activating the brush 140.

In at least one embodiment, device 100 may include one or more covers or trays 143 configured to cover the one or more brushes 140 and collect the first fluid thereon to inhibit the first fluid from falling directly onto the brushes 140.

Device 100 also includes an ultraviolet (UV) light source 150. In at least one embodiment, the UV source light 150 is positioned downstream of the brush 140, and/or downstream of the first sprayer 130.

UV light source 150 is configured to direct UV light onto at least a portion of the exercise mat to disinfect the at least a portion of the exercise mat as the exercise mat passes between the inlet slot 104 of the housing 102 and the outlet slot 108 of the housing 102.

In at least one embodiment, UV light source 150 includes a UV lamp designed to emit UV-C light. It is known that UV-C light is “germicidal” because it can deactivate the DNA of bacteria, viruses and other pathogens and thus destroys their ability to multiply and cause disease. The short wavelength associated with UV-C energy, specifically between about 250 to about 260 nm, and lower, provides the highest germicidal effectiveness, and thus is lethal to a variety or microorganisms, including the most common molds, virus, and bacteria, such as salmonella, staphylococcus, streptococcus, legionella, bacillus, dysentery, infectious hepatitis, influenza, and rotavirus.

In at least one embodiment, UV light source 150 is configured to direct UV-C light onto at least a portion of the exercise mat as the exercise mat travels along the pathway. In at least one embodiment, the UV light source 150 is positioned to direct UV-C light across a width of at least one surface of the exercise mat as the exercise mat travels along the pathway. In at least one embodiment, the UV source 150 includes more than one UV lamp and, together, the more than one UV lamps are positioned to direct UV-C light across a width of two surfaces (e.g. front and back) of the exercise mat as the exercise mat travels along the pathway.

In at least one embodiment, UV light source 150 is communicatively coupled to the processor 170 (described below) to receive control signals from the processor 170. For instance, UV light source 150 may be configured to receive a control signal from the processor 170 activating the UV light source 150 and/or deactivating the UV source 150.

In at least one embodiment, the UV light source 150 is configured to emit UV light having a wavelength and for a period of time to provide for the UV light is disinfect the exercise mat as it passes along the pathway. In at least one embodiment, the first pair of rollers 120 are configured to rotate at a pre-selected speed to provide for the exercise mat to be exposed to the UV light source(s) for a period of time that is sufficient for the UV light sources to disinfect the exercise mat as it passes along the pathway. For instance, in at least one embodiment, the first pair of rollers 120 are configured to rotate at a speed to provide for the mat (e.g. having a length of about 60 inches to about 72 inches, or of about 68 inches) to travel from the first pair of rollers 120 to the outlet slot in a time in a range of about 10 seconds to about 20 seconds, or of about 10 seconds, or of about 20 seconds, or of about 15 seconds.

In at least one embodiment, the device 100 may also includes a second sprayer (not shown) to apply a scent to the exercise mat. In at least one embodiment, second sprayer is positioned downstream of the UV source 150, the first sprayer 130 and the brush 140.

In at least one embodiment, second sprayer is configured to direct a liquid scent onto at least a portion of the exercise mat as the exercise mat passes between the inlet slot 104 of the housing 102 and the outlet slot 108 of the housing 102.

In at least one embodiment, the second sprayer is configured to apply (e.g. spray) a stream of fluid (e.g. liquid or gas) onto at least a portion of the exercise mat to be cleaned. For instance, the second sprayer may be configured to have a width that is equal to or greater than a typical width of an exercise mat (e.g. about two feet, or 24 inches) and spray a linear stream of fluid across a width of the exercise mat as the exercise mat longitudinally passes by the second sprayer.

In another embodiment, the second sprayer may be configured to have a width that is less than or about equal to a typical width of an exercise mat and spray a linear stream of fluid across a portion of the width of the exercise mat as the exercise mat longitudinally passes the second sprayer. In this embodiment, the second sprayer may be movably coupled to the housing 102 and be configured to move laterally in a direction across the width of the exercise mat as the exercise mat passes longitudinally along the pathway.

In at least one embodiment, second sprayer (not shown) may be fluidly coupled to a second reservoir configured to retain the one or more fluids to be applied to the exercise mat. The one or more fluids may be stored as a liquid and may be applied to the exercise mat as a liquid, or, in at least one embodiment, may be aerosolized by the second sprayer and applied to the exercise mat as a gas.

Second sprayer is communicatively coupled to the processor 170 (described below) to receive control signals from the processor 170. For instance, second sprayer may be configured to receive a control signal from the processor 170 activating the second sprayer.

In at least one embodiment, second sprayer may include one or more nozzles configured to apply one or more fluids to the exercise mat simultaneously. For instance, the one or more nozzles may be positioned to apply the one or more fluids to the exercise mat at different angles and from different distances. In at least one embodiment, varying the number of nozzles of second sprayer and/or varying the distance or the contact angle of the fluid as it strikes the exercise mat may optimize the coverage of the fluid on the exercise mat.

Second sprayer is generally used to apply a scent to the exercise mat. The scent may be customizable in that it may include a combination of scents stored in more than one reservoir in the device 100. Applying the scent to the exercise mat may leave it smelling fresh and desirable to use in future exercises.

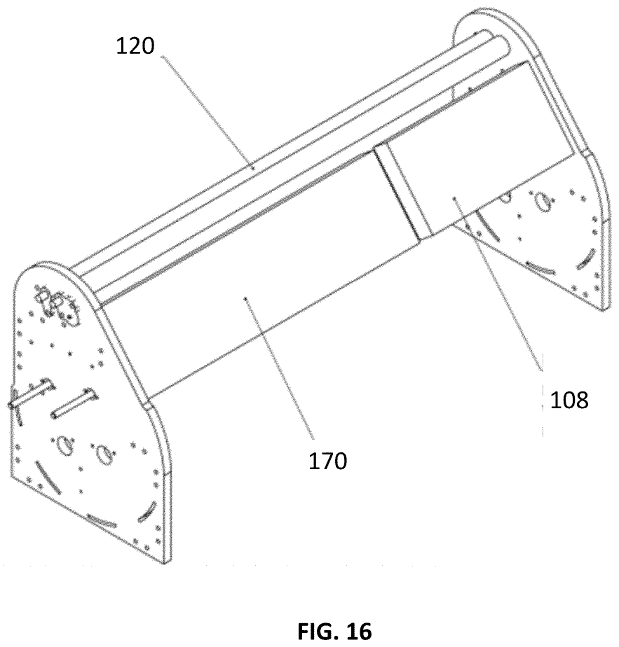

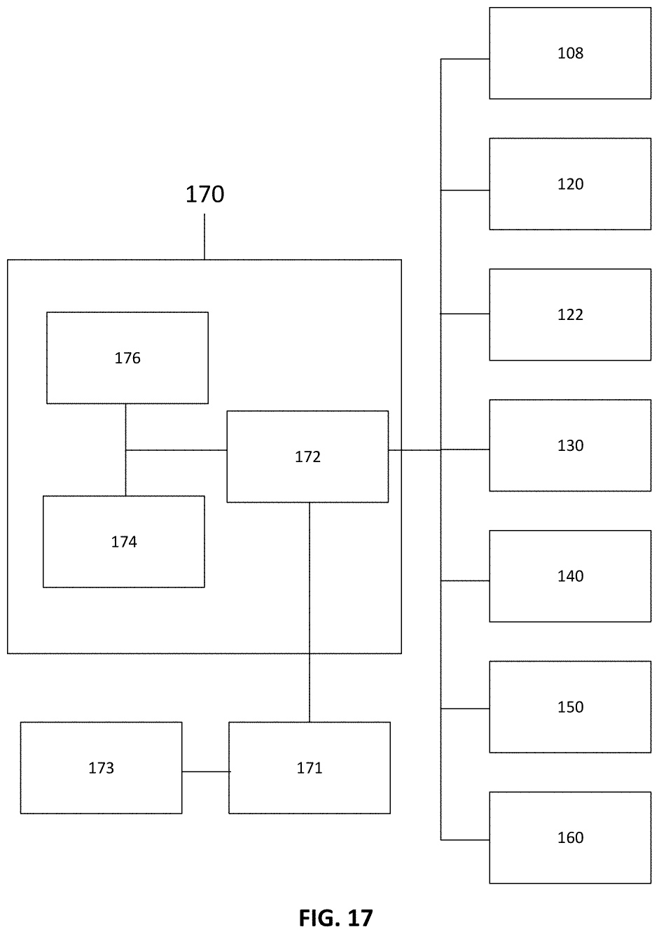

In at least one embodiment, device 100 also includes a processor 170 (see FIGS. 16 and 17) for controlling various components of the device 100. FIG. 17 shows a block diagram of the connections of processor 170 to the various other components of the device 100.

Processor 170 includes a communication interface 172 configured to, for example, provides the processor 170 with connectivity to one or more communication links and/or networks (e.g., LANs, WANs, or wireless LANs) by which the processor may communicate with other devices (e.g. user devices and/or remote servers and/or computing devices).

The communication interface 172 can include any component for facilitating communication with the other components via a communication network 171. For example, the communication interface 172 can include a wireless transceiver for communicating within a wireless communications network. The communication interface 172 can communicate identification data and/or operating data of the devices 100 to the communication network 171. The communication interface 172 can receive commands from the communication network 171.

For example, device 100 may be equipped with a wireless communication interface 172 to enable wireless communications according to a wireless protocol (e.g. near filed communication (NFC), LoRa, Bluetooth®, Bluetooth® Low Energy, Zigbee, or Z-Wave). As noted above, communication interface 172 may transmit information relating to the components of the device 100, including but not limited to the first sprayer 130, the brush 140, the UV source 150, the second sprayer and/or one or more sensor units within the housing 102 to track a position of the exercise mat therein, may also communicate using the communication network.

The communication interface 172 may also facilitate communication between a server 173 and the device 100 via communication network 171.

Processor 170 also includes a storage unit 174 configured to store data for the device 100. Such data may include but is not limited to storage levels with one or more of the reservoirs.

Storage unit 174 may also store the user interface that is presented on the display 108.

Processor 170 also includes a processing unit 176 configured to receive instructions from the communication interface 172 to control the first sprayer 130, the brush 140, the ultraviolet source 150 and/or the second sprayer in cleaning the exercise mat based on the instructions.

In at least one embodiment, processing unit 176 may be configured to receive a payment authorization from a server and/or a mobile device via the communication interface 172. Based on the payment authorization, processing unit 176 may control activation of any one or more of the first sprayer 130, the brush 140, the UV source 150 and/or the second sprayer in cleaning the mat.

Processing unit 176 of processor 170 is also communicatively coupled to the display 108 to control the presentation of the user interface on the display 108.

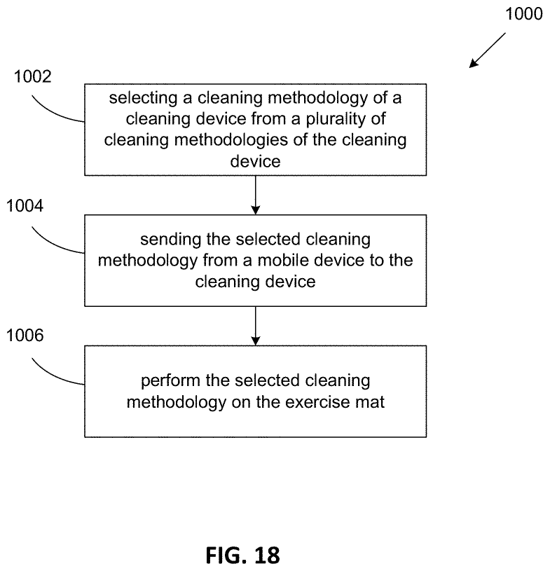

In at least one embodiment, a method 1000 of cleaning an exercise mat is described herein. Method 1000 is shown in FIG. 18. Method 1000 includes at least, at a first step 1002, selecting a cleaning methodology of a cleaning device from a plurality of cleaning methodologies of the cleaning device. The selecting of the cleaning methodology may include but is not limited to selecting one or more cleaning functions of the cleaning device, selecting a disinfecting solution of the cleaning device, selecting a scent of the cleaning device and selecting a coverage area of the exercise mat to be cleaned (e.g. one surface of the exercise mat or two surfaces of the exercise mat).

In at least one embodiment, at a second step 1004, the method includes sending the selected cleaning methodology from a mobile device to the cleaning device. In at least one embodiment, the sending the selected cleaning methodology from a mobile device to the cleaning device includes sending a payment authorization from the mobile device to the cleaning device. In at least one embodiment, the cleaning device may receive the payment authorization from a server rather than from the mobile device.

In at least one embodiment, an application stored on the mobile device can interface with the cleaning device using near-field communication (NFC), which, in at least one embodiment, may be implemented using Apple's Core NFC SDK, for example. Once a user is ready to pay for a cleaning methodology, the user may open the application on their mobile device, select a payment plan within the application on the mobile device and hold their mobile device in proximity to the cleaning device. In at least one embodiment, the user may be asked to verify their identity (e.g. thumbprint, face-ID, or password). If the verification is successful, the application will transmit a user verification message to the cleaning device and the cleaning device will begin the cleaning methodology.

In at least one embodiment, at a third step 1006, the cleaning device may perform the selected cleaning methodology on the exercise mat.

Following the performant of the cleaning methodology by the cleaning device, the cleaning device may transmit a receipt to an email address of the user. In at least one embodiment, the server may send the receipt to the email address of the user upon receiving a confirmation from the cleaning device that the selected cleaning methodology has been completed.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

While the above description provides examples of one or more apparatus, methods, or systems, it will be appreciated that other apparatus, methods, or systems may be within the scope of the claims as interpreted by one of skill in the art.

What is claimed is:

1. A device for cleaning an exercise mat, the device comprising:
a housing;
a first pair of rollers positioned within the housing and being configured to receive the exercise mat through an inlet slot of the housing and direct the exercise mat towards an outlet slot of the housing;
a first sprayer positioned downstream of the first pair of rollers and configured to apply a first fluid to at least one of two opposed surfaces of the exercise mat as the exercise mat passes between the inlet slot of the housing and the outlet slot of the housing;
a brush positioned downstream of the first sprayer and configured to impact the exercise mat to remove contaminants from the exercise mat after the first sprayer has applied the first fluid as the exercise mat passes between the inlet slot of the housing and the outlet slot of the housing; and
an ultraviolet light source configured to direct ultraviolet light onto a first surface and a second surface of the two opposed surfaces of the exercise mat to disinfect each of the first surface and the second surface after the brush has impacted the exercise mat.

2. The device of claim 1 further comprising a storage reservoir to store the first fluid and a pump to provide the first fluid from the storage reservoir to the first sprayer.

3. The device of claim 2, wherein the pump is communicatively connected to a door covering the inlet slot such that the pump is activated to provide the first fluid to the first sprayer when the door to the inlet slot is open.

4. The device of claim 3, wherein the pump is communicatively connected to the door covering the inlet slot such that the pump is deactivated when the door to the inlet slot is closed.

5. The device of claim 1 further comprising a second set of rollers positioned within the housing downstream from the first set of rollers, the second set of rollers being configured to receive the exercise mat from the first set of rollers and direct the exercise mat towards an outlet slot of the housing.

6. The device of claim 1 further comprising:
a processor comprising:
a communication interface; and
a processing unit configured to receive instructions from the communication interface and control the first sprayer, the brush and/or the ultraviolet source based on the instructions from the communication interface.

7. The device of claim 6, wherein the processing unit is configured to, in response to receiving a payment authorization from the communication interface, activate the first sprayer, the brush and/or the ultraviolet source.

8. The device of claim 7, wherein the communication interface is configured to transmit a payment request to a payment server and receive the payment authorization from the payment server.

9. The device of claim 1, wherein the first sprayer includes one or more nozzles positioned to direct the first fluid onto the exercise mat at differing angles.

10. The device of claim 1, wherein the first sprayer is positioned to direct a fluid onto the exercise mat as the exercise mat is bent to expose grooves of the exercise mat and provide for the first fluid to enter the grooves of the exercise mat.

11. The device of claim 1, wherein the first fluid is a disinfectant.

12. The device of claim 1 further comprising a tray positioned below the first sprayer, the tray configured to collect at least a portion of the first fluid.

13. The device of claim 12, wherein the tray is removable from the housing.

14. The device of claim 2, wherein the storage reservoir is positioned outside of the housing.

15. The device of claim 2, wherein the storage reservoir is positioned inside of the housing.
